(19) United States
(12) Patent Application Publication
(10) Pub. No.: US 2018/0071034 A1
(43) Pub. Date: Mar. 15, 2018

(54) MEDICAL MANIPULATOR SYSTEM

(71) Applicant: OLYMPUS CORPORATION, Tokyo (JP)

(72) Inventor: Takumi ISODA, Tokyo (JP)

(73) Assignee: OLYMPUS CORPORATION, Tokyo (JP)

(21) Appl. No.: 15/815,974

(22) Filed: Nov. 17, 2017

Related U.S. Application Data
(63) Continuation of application No. PCT/JP2016/065607, filed on May 26, 2016.

(30) Foreign Application Priority Data
May 27, 2015 (JP) 2015-107480

(51) Int. Cl. A61B 34/00; A61B 90/00; A61B 1/00

(52) U.S. Cl. CPC A61B 34/25; A61B 34/20; A61B 1/00133; A61B 90/37

(57) ABSTRACT

A medical manipulator system including an endoscope; a manipulator, the manipulator comprising a through-hole configured to hold the endoscope, wherein the manipulator is configured to move the endoscope; an operating unit configured to output an operating command; and a controller comprising one or more processors, the one or more processors configured to: acquire an image of the endoscope in a first configuration, the first configuration being a configuration of the endoscope at least partially inserted into the through-hole, the image comprising an inner surface of the through-hole; recognize a type of the endoscope based on the image; assign the endoscope to a recognized type of endoscope; receive an operating command from the operating unit; change the operating command based on the recognized type of endoscope to a changed operating command; and control the manipulator under the changed operating command.

### CROSS-REFERENCE TO RELATED APPLICATIONS

This is a Continuation Application of International Application No. PCT/JP2016/065607 filed on May 26, 2016, which claims priority to Japanese Application No. 2015-107480 filed on May 27, 2015. The contents of International Application No. PCT/JP2016/065607 and Japanese Application No. 2015-107480 are hereby incorporated herein by reference in its entirety.

### TECHNICAL FIELD

The present invention relates to a medical manipulator system.

### BACKGROUND ART

There is a known endoscope apparatus in which, each time a different type of endoscope is mounted on the apparatus main body provided with an image capturing element and a light source, the apparatus main body reads out property information from a memory provided as a property information storing unit in the endoscope to store the property information of the endoscope and then sets the property information.

### SUMMARY OF INVENTION

One aspect of the present disclosure is a medical manipulator system including an endoscope; a manipulator, the manipulator comprising a through-hole configured to hold the endoscope, wherein the manipulator is configured to move the endoscope; an operating unit configured to output an operating command; and a controller comprising one or more processors, the one or more processors configured to: acquire an image of the endoscope in a first configuration, the first configuration being a configuration of the endoscope at least partially inserted into the through-hole, the image comprising an inner surface of the through-hole; recognize a type of the endoscope based on the image; assign the endoscope to a recognized type of endoscope; receive an operating command from the operating unit; change the operating command based on the recognized type of endoscope to a changed operating command; and control the manipulator under the changed operating command.

The disclosure is also directed to a method for controlling medical manipulator system, the method comprising:

acquiring an image from an endoscope when the endoscope in a first configuration, the first configuration being a configuration of the endoscope at least partially inserted into the through-hole of distal end of a maniplulator, the image comprising an inner surface of the through-hole;

recognizing a type of the endoscope based on the image;

assigning the endoscope to a recognized type of endoscope;

receiving an operating command from an operating unit;

changing the operating command based on the recognized type of the endoscope to a changed operating command; and

controlling the manipulator under the changed operating command.

The disclosure is also directed to a method for controlling medical manipulator system, wherein the image comprises a predetermined target on the inner surface of the through-hole, and the method comprising:

recognizing the type of the endoscope as the recognized type of endoscope based on the position of the predetermined target in the image.

The disclosure is also directed to a for controlling medical manipulator system, wherein the predetermined target is an opening of the manipulator, through the through-hole in the image.

The disclosure is also directed to a method for controlling medical manipulator system according, the method comprising:

acquiring a plurality of the images from the endoscope from the first configuration to a second configuration, the second configuration being a configuration of the endoscope at least partially inserted into the through-hole, the second configuration of the endoscope being a configuration of further insertion into the through hole as compared of the first configuration, at least one of the plurality of images comprising an inner surface of the through-hole;

recognizing the recognized type of the endoscope based on a trajectory of the predetermined target in the plurality of the images.

The disclosure is also directed to a method for controlling medical manipulator system, the method comprising:

changing the operating command based on the recognized type of the endoscope so as to move an optical axis of the endoscope in the same direction of the operating command input by the operating unit.

The disclosure is also directed to a method for controlling medical manipulator system, the method comprising:

acquiring a time-variation of an outer surface of an endoscope in the radial direction of the endoscope from a sensor of a manipulator, the sensor configured to sequentially detect a radial position of an outer surface of the endoscope from a first configuration to a second configuration, the first configuration being a configuration of the endoscope at least partially inserted into the through-hole, the second configuration being a configuration of the endoscope at least partially inserted into the through-hole the second configuration of the endoscope being a configuration of further insertion into the through hole as compared to the first configuration;

recognizing a type of the endoscope based on sequential images;

receiving an operating command from an operating unit;

changing the operating command based on the recognized type of the endoscope to a changed operating command; and

controlling the manipulator under the changed operating command.

The disclosure is also directed to a method for controlling medical manipulator, wherein the sensor configured to detect an amount of insertion of the endoscope in the through-hole and a distance between the outer surface of the endoscope and the sensor.

The disclosure is also directed to a method for controlling medical manipulator, the method comprising:

changing the operating command based on the recognized type of the endoscope so as to move an optical axis of the endoscope in the same direction of the operating command input by the operating unit.

### BRIEF DESCRIPTION OF DRAWINGS

FIG. 1 is an overall configuration diagram showing a medical manipulator system according to one embodiment of the present invention.

FIG. 2 is a perspective view schematically showing a holder for mounting an endoscope probe on a manipulator of the medical manipulator system in FIG. 1.

FIG. 3 is a block diagram showing a controller of the medical manipulator system in FIG. 1.

FIG. 4A is a diagram showing an example image inside a through-hole of the holder, acquired by a direct-viewing-type endoscope probe of the medical manipulator system in FIG. 1.

FIG. 4B is a diagram showing an example image inside the through-hole of the holder, acquired by an oblique-viewing-type endoscope probe (with a small oblique angle) of the medical manipulator system in FIG. 1.

FIG. 4C is a diagram showing an example image inside the through-hole of the holder, acquired by an oblique-viewing-type endoscope probe (with a large oblique angle) of the medical manipulator system in FIG. 1.

FIG. 5A is a diagram showing a manipulator on which an oblique-viewing-type endoscope probe is mounted in the medical manipulator system of FIG. 1.

FIG. 5B is a diagram showing an example image acquired with an endoscope probe in the medical manipulator system of FIG. 1.

FIG. 5C is a diagram showing the manipulator on which a direct-viewing-type endoscope probe is mounted in the medical manipulator system of FIG. 1.

FIG. 6A is a diagram of a modification of FIG. 4A, showing an example image inside the through-hole of the holder, acquired by a direct-viewing-type endoscope probe.

FIG. 6B is a diagram of a modification of FIG. 4B, showing an example image inside the through-hole of the holder, acquired by an oblique-viewing-type endoscope probe (with a small oblique angle).

FIG. 6C is a diagram of a modification of FIG. 4C, showing an example image inside the through-hole of the holder, acquired by an oblique-viewing-type endoscope probe (with a large oblique angle).

FIG. 7A is a diagram illustrating a difference in distance between the marks in the image of FIG. 6A when using a direct-viewing-type endoscope probe.

FIG. 7B is a diagram illustrating a difference in distance between the marks in the image of FIG. 6B when using an oblique-viewing-type endoscope probe (with a small oblique angle).

FIG. 7C is a diagram illustrating a difference in distance between the marks in the image of FIG. 6C when using an oblique-viewing-type endoscope probe (with a large oblique angle).

FIG. 8 is a perspective view showing a modification of the processing for recognizing the type of an endoscope probe.

FIG. 9A is a diagram showing a movement trajectory of a mark in the through-hole of the holder, acquired by a direct-viewing-type endoscope probe, resulting from the processing in FIG. 8.

FIG. 9B is a diagram showing a movement trajectory of the mark in the through-hole of the holder, acquired by an oblique-viewing-type endoscope probe (with a small oblique angle), resulting from the processing in FIG. 8.

FIG. 9C is a diagram showing a movement trajectory of the mark in the through-hole of the holder, acquired by an oblique-viewing-type endoscope probe (with a large oblique angle), resulting from the processing in FIG. 8.

FIG. 10 is a longitudinal sectional view showing a trocar, as a modification of the insertion receiving member in FIG. 2.

FIG. 11A is a diagram illustrating processing for inserting an oblique-viewing-type endoscope probe into the trocar in FIG. 10.

FIG. 11B is a diagram showing an output waveform of a distance sensor acquired via the process of inserting the endoscope probe shown in FIG. 11A.

FIG. 12A is a diagram illustrating the process of inserting a direct-viewing-type endoscope probe into the trocar in FIG. 10.

FIG. 12B is a diagram showing an output waveform of the distance sensor, acquired via the process of inserting a direct-viewing-type endoscope probe shown in FIG. 12A.

FIG. 13A is a diagram illustrating process of inserting an oblique-viewing-type endoscope probe into a modification of the trocar in FIG. 10.

FIG. 13B is a diagram showing the relationship between the insertion amount of the endoscope probe and the output of the distance sensor acquired via the process of inserting the oblique-viewing-type endoscope probe shown in FIG. 13A.

### DESCRIPTION OF EMBODIMENTS

A medical manipulator system 1 according to one embodiment of the present invention will now be described with reference to the drawings.

In the discussion and claims herein, the term “about” indicates that the value listed may be somewhat altered, as long as the alteration does not result in nonconformance of the process or structure to the illustrated embodiment. For example, for some elements the term “about” can refer to a variation of ±0.1%, for other elements, the term “about” can refer to a variation of ±1% or ±10%, or any point therein.

As used herein, the term “substantially”, or “substantial”, is equally applicable when used in a negative connotation to refer to the complete or near complete lack of an action, characteristic, property, state, structure, item, or result. For example, a surface that is “substantially” flat would either be completely flat, or so nearly flat that the effect would be the same as if it were completely flat.

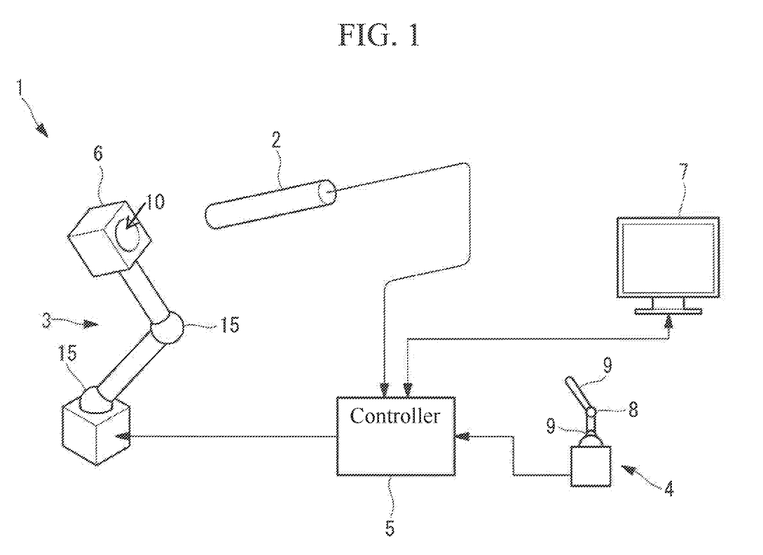

As shown in FIG. 1, the medical manipulator system 1 according to this embodiment includes: an endoscope 2; a manipulator 3 that holds this endoscope 2 at a distal end thereof and that moves the endoscope 2; an operating unit 4 for allowing an operator to input an operating command; a controller 5 for controlling the manipulator 3 on the basis of the operating command input to the operating unit 4; a holder (insertion receiving member) 6 that is fixed to the distal end of the manipulator 3 and on which the endoscope 2 is mounted; and a monitor 7 for displaying the image acquired by the endoscope 2.

The endoscope 2 has an image sensor (not shown in the figure) at the distal end thereof and is capable of acquiring an image of a subject. The endoscope 2 is formed in an elongated shape. The types of the endoscope 2 include, and are not limited to, a direct-viewing type, in which a front image of an anterior direction in the longitudinal axis direction is acquired, and an oblique-viewing type, in which an image of an anterior oblique direction is acquired, with endoscopes of different oblique angles available for the oblique-viewing type.

The manipulator 3 has, for example, a multi-joint structure and has a sufficiently high degree of freedom to set the position/orientation of the holder 6 fixed to the distal end thereof.

The operating unit 4 also has, for example, a multi-joint structure similar or substantially similar to that of the manipulator 3 and detects, as a motion command, the angle of each joint 9 for achieving the position/orientation of a handle 8 gripped and operated by the operator and outputs the motion command to the controller 5.

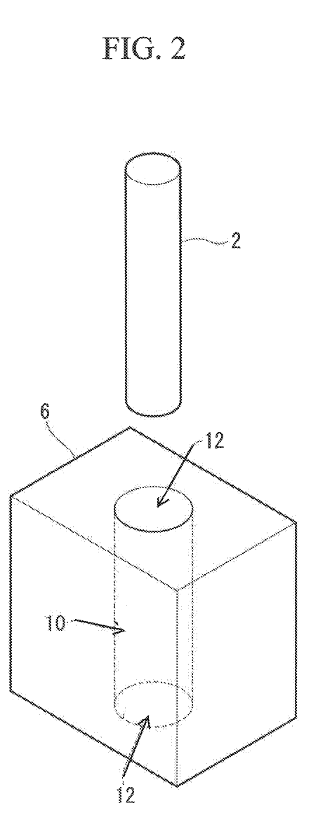

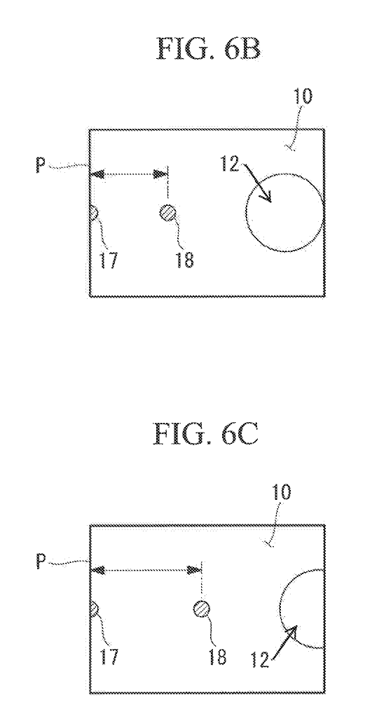

As shown in FIG. 2, the holder 6 includes a through-hole 10 into which the endoscope 2 is inserted to mount the endoscope 2 on the manipulator 3 and also a fixing mechanism, not shown in the figure, for fixing the endoscope 2 inserted into this through-hole 10. Furthermore, on the inner surface of the through-hole 10 of the holder 6, a mark for recognizing the type of the endoscope 2 with a scope-type recognition unit 11, to be described later in the discussion of FIG. 6A (for example), can be provided. The mark may have any shape, and an opening 12 itself of the through-hole 10 formed in the holder 6 may be used as the mark.

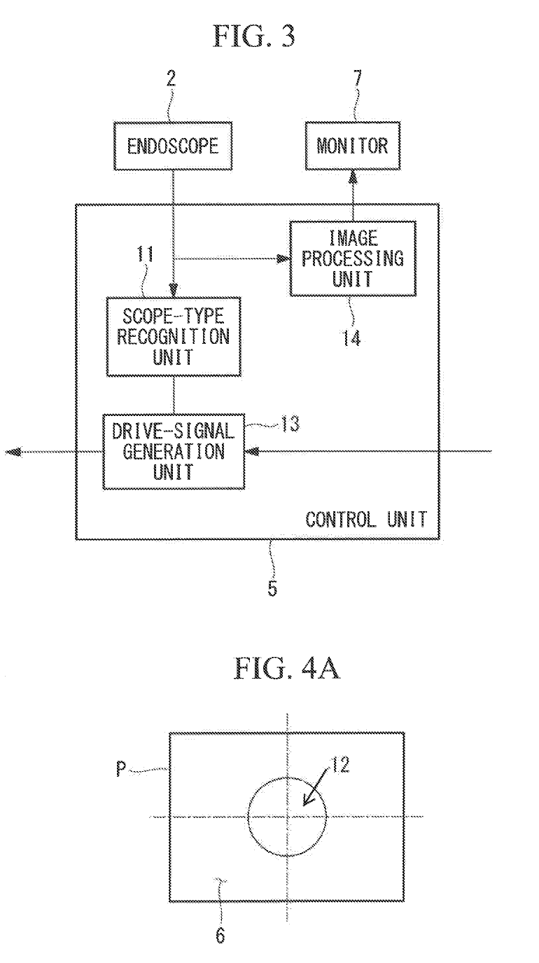

As shown in FIG. 3, the controller 5 includes: a drive-signal generation unit (control unit) 13 that receives the motion command sent from the operating unit 4, generates a drive command signal for driving each joint 15 of the manipulator 3, and outputs the signal to the manipulator 3; the scope-type recognition unit 11 for recognizing the type of the endoscope 2 on the basis of an image that is acquired by the image-capturing unit when the endoscope 2 is inserted into the through-hole 10 of the holder 6; and an image processing unit 14 for processing the image acquired by the image-capturing unit and displaying the image on the monitor 7.

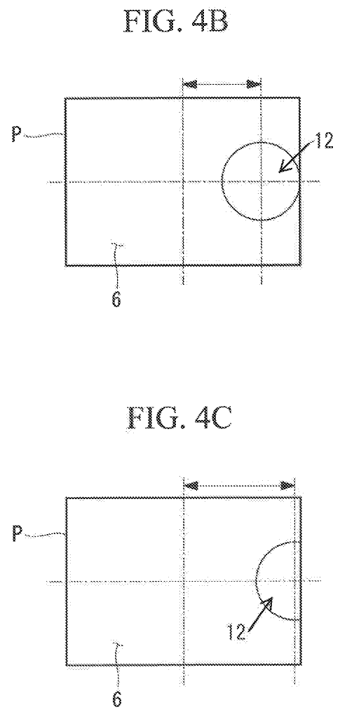

The scope-type recognition unit 11 recognizes the type of the endoscope 2 as the direct-viewing type when an image P that is acquired by the image-capturing unit when the endoscope 2 is inserted into the through-hole 10 of the holder 6 shows the opening 12 of the through-hole 10 at about the center of the image P, as shown in FIG. 4A. The scope-type recognition unit 11 recognizes the type of the endoscope 2 as the oblique-viewing type when the image P that is acquired by the image-capturing unit when the endoscope 2 is inserted into the through-hole 10 of the holder 6 shows the opening 12 of the through-hole 10 at a position shifted in one direction relative to the center of the image P, as shown in FIGS. 4B and 4C.

Furthermore, if the amount of shift of the opening 12 of the through-hole 10 relative to the center of the image P, as detected when the endoscope 2 is inserted into the through-hole 10 of the holder 6 by a prescribed depth, varies as shown in FIG. 4B and FIG. 4C, then the scope-type recognition unit 11 recognizes that the larger the amount of shift, the larger the oblique angle of the optical axis of the image-capturing unit relative to the longitudinal axis of the endoscope 2. For example, the oblique angle is about 30° in the case of FIG. 4B, and the oblique angle is about 45° in the case of FIG. 4C.

The drive-signal generation unit 13 receives information about the type of the endoscope 2 recognized by the scope-type recognition unit 11 and switches the motion of the manipulator 3.

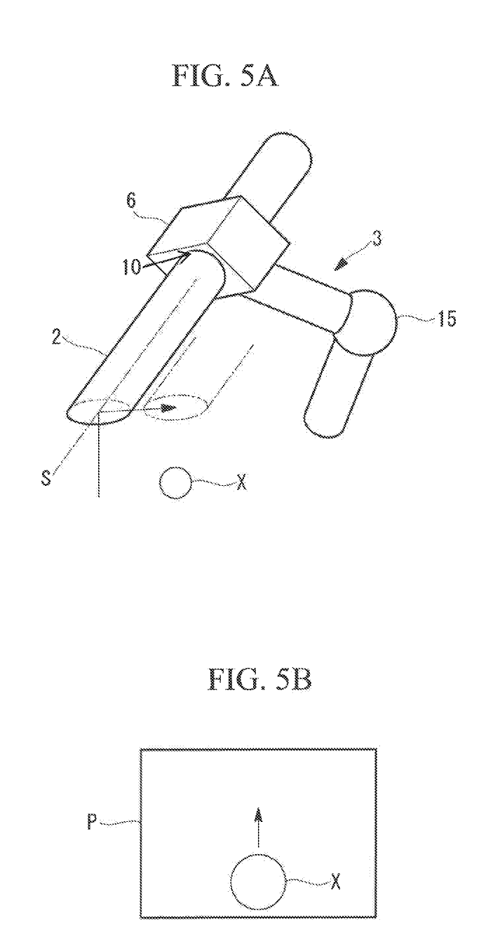

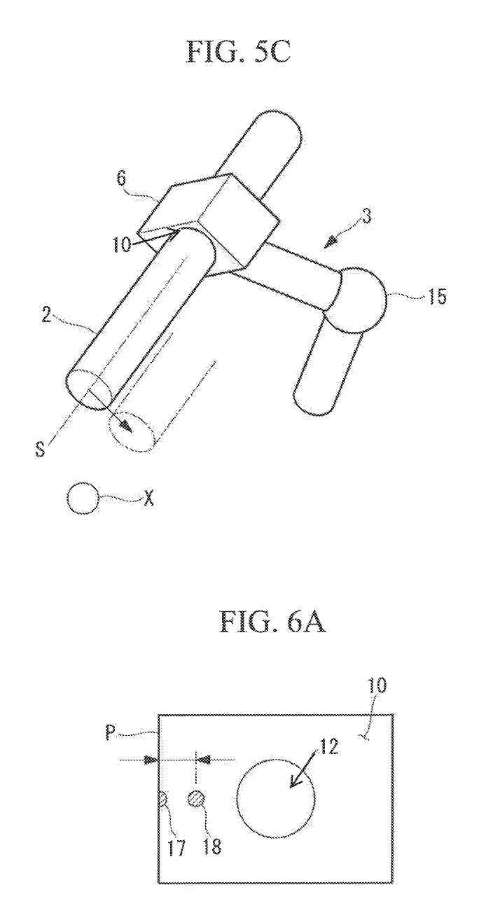

For example, if the endoscope 2 fixed to the holder 6 is of the oblique-viewing type, as shown in FIG. 5A, and the image P of a subject X as shown in FIG. 5B is acquired and displayed on the monitor 7, then the distal end of the endoscope 2 is moved in a direction orthogonal to an optical axis S in FIG. 5A, as indicated by the arrow, in order to move the subject X in a direction as indicated by the arrow on the image P.

On the other hand, if the endoscope 2 fixed to the holder 6 is of the direct-viewing type, as shown in FIG. 5C, and the image P of the subject X as shown in FIG. 5B is acquired and displayed on the monitor 7, then the distal end of the endoscope 2 is moved in a direction orthogonal to the optical axis S in FIG. 5C, as indicated by the arrow, in order to move the subject X in a direction indicated by the arrow on the image P. The movement direction in this case differs from that in the case of FIG. 5A, and the motion of the manipulator 3 for achieving this movement differs accordingly.

The drive-signal generation unit 13 is configured to control the manipulator 3, by switching the motion of the manipulator 3 according to the type of the endoscope 2, so that the subject X in the image P acquired by the endoscope 2 makes the same movement by a substantially similar operation performed by the operator via the operating unit 4.

The operation of the medical manipulator system 1 according to this embodiment with the above-described configuration will be described below.

In order to observe the interior of the body of a patient using the medical manipulator system 1 according to this embodiment, first the power supply of the endoscope 2 is turned on, and then in a state where the image P can be acquired by the image-capturing unit provided at the distal end of the endoscope 2, the endoscope 2 is inserted by a prescribed amount from the distal end side into the through-hole 10 of the holder 6 provided at the distal end of the manipulator 3.

Then, the image P, including the inner surface of the through-hole 10, is acquired by the image-capturing unit, showing the opening 12 of the through-hole 10 of the holder 6 in the image P. As a result of this image P being sent to the scope-type recognition unit 11, the position of the opening 12 in the image P is calculated by image processing. If the center position of the opening 12 is disposed within a predetermined range from the center of the image as a result of image processing, then the type of the endoscope 2 is recognized as the direct-viewing type. Furthermore, if the center position of the opening 12 is shifted from the center of the image as a result of image processing, then the type of the endoscope 2 is recognized as the oblique-viewing type, and the oblique angle is recognized according to the amount of shift. Thereafter, the result of recognition in the scope-type recognition unit 11 is sent to the drive-signal generation unit 13.

In the drive-signal generation unit 13, the control of the manipulator 3 is switched on the basis of the received information about the type of the endoscope 2.

Subsequently, the endoscope 2 inserted into the through-hole 10 of the holder 6, is fixed to the holder 6, and then the manipulator 3 is driven as a result of the operator operating the operating unit 4, thereby allowing the position of the subject X in the image P, which has been acquired by the endoscope 2 and displayed on the monitor 7, to be changed.

In this case, according to the medical manipulator system 1 of this embodiment, since control of the manipulator 3 is switched on the basis of information about the type of the endoscope 2, the operator, without having to take into consideration the type of the mounted endoscope 2, can move the position of the image P displayed on the monitor 7 in the same direction by applying a substantially similar operation to the operating unit 4.

In this manner, the medical manipulator system 1 according to this embodiment affords an advantage in that the operability can be enhanced because the operator does not need to change the operation applied to the operating unit 4 depending on the type of the endoscope 2. In addition, the operability can be enhanced in the same manner with an existing endoscope 2 because the type of the endoscope 2 is recognized on the basis of the image P acquired by the endoscope 2, instead of making the endoscope 2 itself store identification information thereof.

This embodiment has been described by way of an example where control is automatically switched according to the type of the endoscope 2. Alternatively, the recognized type of the endoscope 2 may be displayed to the operator so that the operator can change the operation to be performed according to the recognized type.

The opening 12 of the through-hole 10 of the holder 6 is used as a mark in the medical manipulator system 1 according to this embodiment. In other embodiments, dot-shaped marks 16 may be provided on the inner surface of the through-hole 10.

Further, as shown in FIGS. 6A to 6C, two dot-shaped marks 17 and 18 spaced apart from each other in the longitudinal direction of the through-hole 10 may be provided, so that the type of the endoscope 2 can be recognized, for example, as the direct-viewing type, as the 30° oblique-viewing type, or as the 45° oblique-viewing type, according to the distance between the marks 17 and 18 as detected when the mark 17 on the near side is disposed at an edge of the image. In this embodiment, marks 17 and 18 are shown as being circular, but, in other embodiments, marks can be any suitable shape and/or size.

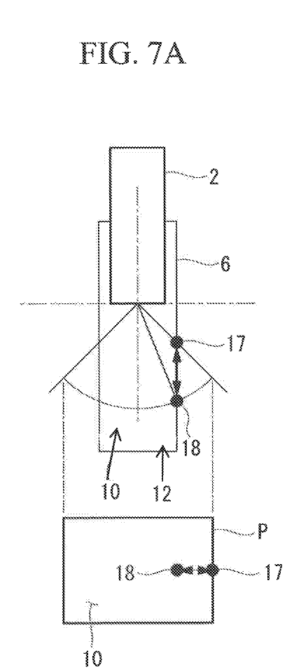

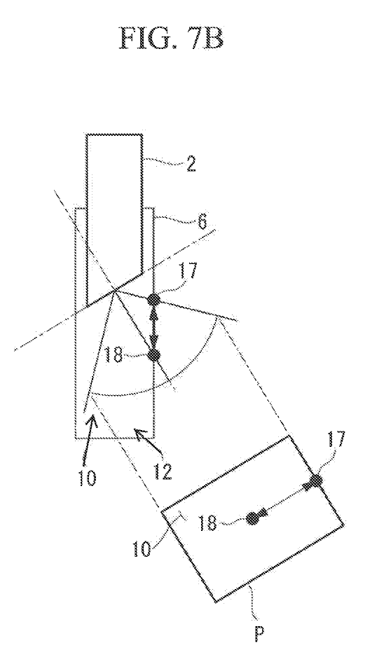

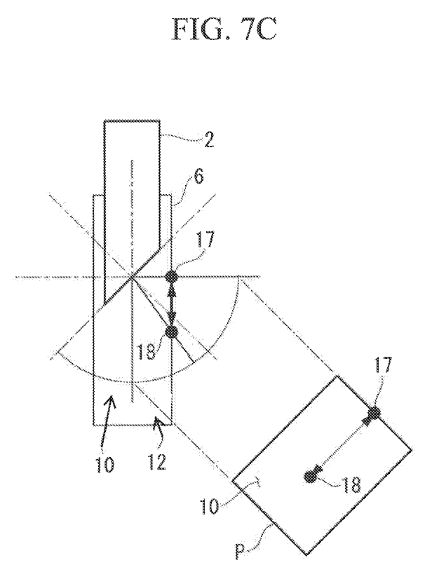

More specifically, as shown in FIGS. 7A to 7C, the distance between the marks 17 and 18 as imaged appears to be the smallest for the endoscope 2 of the direct-viewing type and increases in the order of the 30° oblique-viewing type and the 45° oblique-viewing type. Thus, the type of the endoscope 2 can be recognized by detecting the viewed distance between the marks 17 and 18.

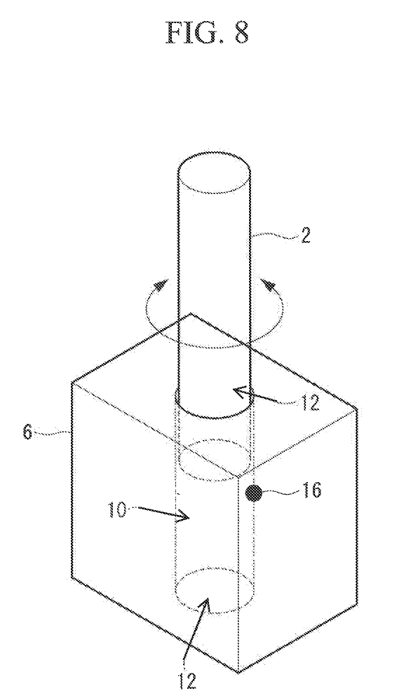

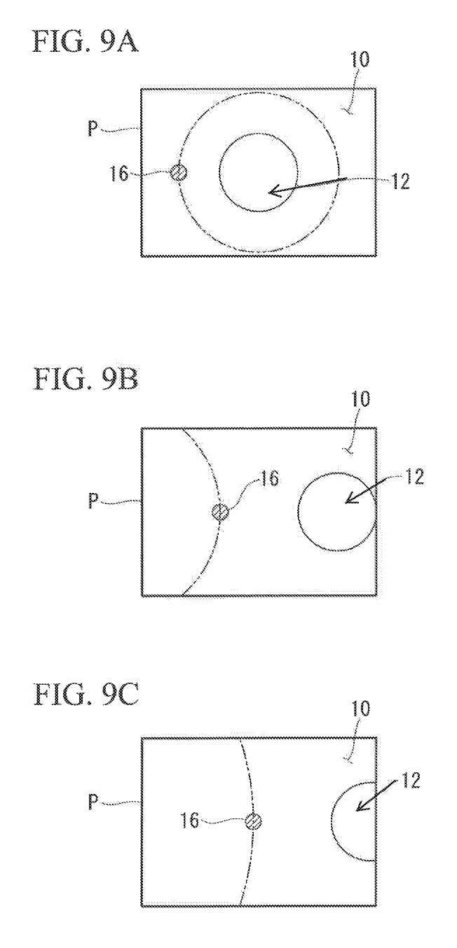

Furthermore, as shown in FIG. 8 and FIGS. 9A to 9C, as a result of the endoscope 2 being rotated about the longitudinal axis of the endoscope 2 in a state where the endoscope 2 is inserted into the through-hole 10 of the holder 6, the dot-shaped mark 16 on the inner surface of the through-hole 10 moves in acquired images with an arc-shaped movement trajectory. Therefore, detecting the radius of this movement trajectory also makes it possible to recognize the type of the endoscope 2.

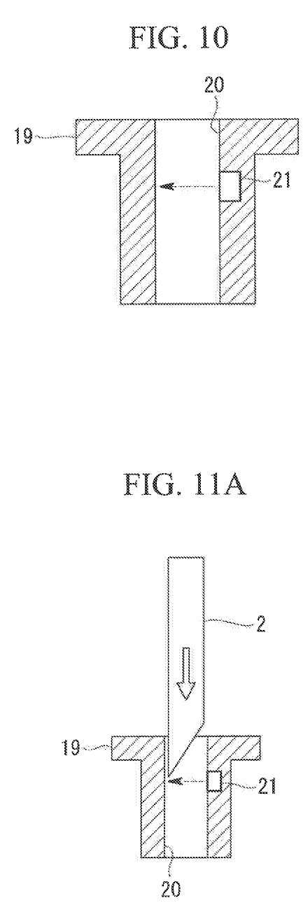

In this embodiment, the holder 6 provided with the through-hole 10 into which the endoscope 2 can be inserted when the endoscope 2 is to be mounted on the manipulator 3 has been exemplified as the insertion receiving member. In other embodiments, as shown in FIG. 10, a trocar 19 that can be mounted in the body surface tissue of the patient may be used.

In the example shown in FIG. 10, the trocar 19 is provided with a through-hole 20 into which the endoscope 2 can be inserted and also a distance sensor (outer-surface-position detection unit) 21, on the inner surface of the through-hole 20, that measures the distance from the distance sensor 21 surface to either the opposing surface of the through-hole 20 or the outer surface of the inserted endoscope 2.

An example of the distance sensor 21 is an optical sensor that emits light and receives reflected light that is reflected.

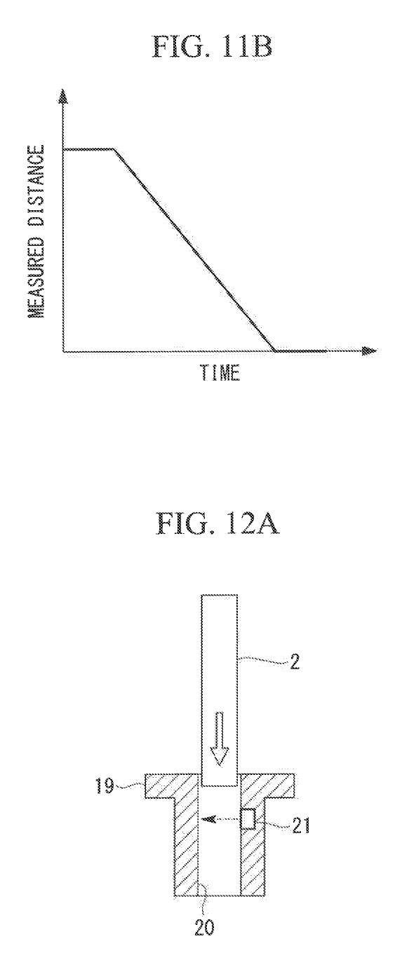

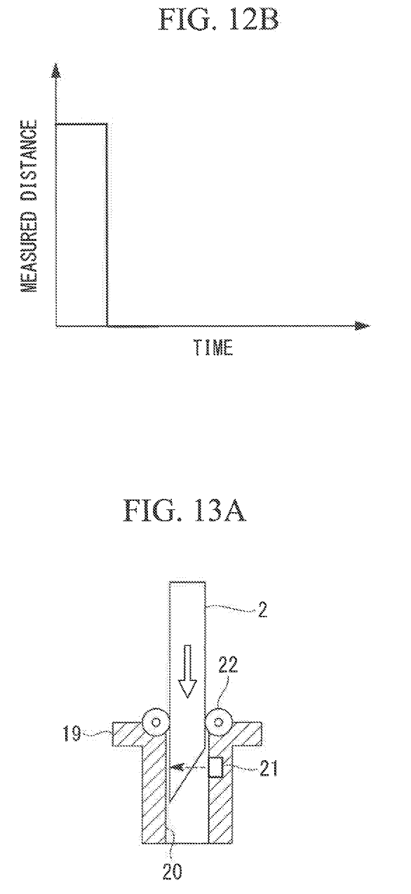

In this manner, since the distance from the inner surface of the through-hole 20 to the outer surface of the endoscope 2 is detected sequentially as the distal end of the endoscope 2 is inserted into the through-hole 20 of the trocar 19, the type of the endoscope 2 can be recognized as the oblique-viewing type if the distance changes relatively gradually, as shown in FIGS. 11A and 11B, or as the direct-viewing type if the distance changes relatively abruptly, as shown in FIGS. 12A and 12B.

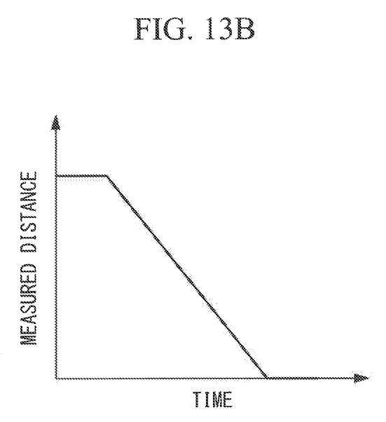

Furthermore, the trocar 19 may be provided with an encoder 22 for detecting the amount of insertion of the endoscope 2 as shown in FIG. 13A, so that the oblique angle of the oblique-viewing type can be determined on the basis of the relationship between the amount of insertion detected by the encoder (insertion-amount detection unit) 22 and the distance detected by the distance sensor 21. As shown in FIG. 13B, the oblique angle of the endoscope 2 of the oblique-viewing type can be recognized from the slope of the graphical relationship representing distance vs. amount of insertion.
1 Medical manipulator system
2 Endoscope
3 Manipulator
4 Operating unit
5 Controller
6 Holder (insertion member, holding section)
10, 20 Through-hole
11 Scope-type recognition unit
12 Opening (mark)
16, 17, 18 Mark
19 Trocar (insertion member)
21 Distance sensor (outer-surface-position detection unit)
22 Encoder (insertion-amount detection unit)

1. A medical manipulator system comprising:
an endoscope;
a manipulator, the manipulator comprising a through-hole configured to hold the endoscope, wherein the manipulator is configured to move the endoscope;
an operating unit configured to output an operating command; and
a controller comprising one or more processors, the one or more processors configured to:
acquire an image of the endoscope in a first configuration, the first configuration being a configuration of the endoscope at least partially inserted into the through-hole, the image comprising an inner surface of the through-hole;
recognize a type of the endoscope based on the image;
assign the endoscope to a recognized type of endoscope;
receive an operating command from the operating unit;
change the operating command based on the recognized type of endoscope to a changed operating command; and
control the manipulator under the changed operating command.

2. The medical manipulator system according to claim 1, wherein the image comprises a predetermined target on the inner surface of the through-hole, and
the processor is further configured to recognize the type of the endoscope as the recognized type of endoscope based on the position of the predetermined target in the image.

3. The medical manipulator system according to claim 2, wherein the predetermined target is an opening of the manipulator, through the through-hole, in the image.

4. The medical manipulator system according to claim 2, wherein the processor is further configured to:
acquire a plurality of images of the endoscope from the first configuration to a second configuration, the second configuration being a configuration of the endoscope at least partially inserted into the through-hole, the second configuration of the endoscope being a configuration of further insertion into the through hole as compared of the first configuration, at least one of the plurality of images comprising an inner surface of the through-hole;
recognize the recognized type of the endoscope based on a trajectory of the predetermined target in the plurality of the images.

5. The medical manipulator system according to claim 1, wherein the processor is further configured to change the operating command based on the recognized type of the endoscope so as to move an optical axis of the endoscope in the same direction of the operating command input by the operating unit.

6. A medical manipulator system comprising:
an endoscope;
a manipulator, the manipulator comprising a through-hole configured to hold the endoscope, wherein the manipulator is configured to move the endoscope;
a sensor configured to sequentially detect a radial position of an outer surface of the endoscope from a first configuration to a second configuration, the first configuration being a configuration of the endoscope at least partially inserted into the through-hole, the second configuration being a configuration of the endoscope at least partially inserted into the through-hole the second configuration of the endoscope being a configuration of further insertion into the through hole as compared to the first configuration;
an operating unit configured to output an operating command; and
a controller comprising one or more processors, the one or more processors configured to:
acquire sequential images of an outer surface of the endoscope when the endoscope is moved from the first configuration to the second configuration;
recognize a type of the endoscope based on the sequential images;
assign the endoscope to a recognized type of endoscope;
receive an operating command from the operating unit;
change the operating command based on the recognized type of the endoscope to a changed operating command; and
control the manipulator under the changed operating command.

7. The medical manipulator system according to claim 6, wherein the sensor is configured to detect an amount of insertion of the endoscope in the through-hole and a distance between the outer surface of the endoscope and the sensor.

8. The medical manipulator system according to claim 6, wherein the processor is further configured to change the operating command based on the recognized type of the endoscope so as to move an optical axis of the endoscope in the same direction of the operating command input by the operating unit.
